(19) United States
(12) Patent Application Publication
(10) Pub. No.: US 2008/0269475 A1
(43) Pub. Date: Oct. 30, 2008

(54) Sorbent for Nucleic Acids, Comprising Acid-Activated Layer Silicate

(76) Inventor: Ulrich Sohling, Freising (DE)

Correspondence Address: SCOTT R. COX;LYNCH, COX, GILMAN & MAHAN, P.S.C., 500 WEST JEFFERSON STREET, SUITE 2100, LOUISVILLE, KY, 40202, US

(21) Appl. No.: 11/629,044

(22) PCT Filed: Jun. 10, 2005
(86) PCT No.: PCT/EP2005/006250

§ 371 Date: Jan. 12, 2007

(30) Foreign Application Priority Data
Jun. 10, 2004 (DE) 10 2004 028 267.6

(51) Int. Cl. C07H 1/00; B01J 20/00

(52) U.S. Cl. 536/25.4; 502/404

(57) ABSTRACT

The invention relates to a method for removing or recovering at least one nucleic acid molecule from an aqueous or alcoholic medium with the aid of a sorbent, where the sorbent includes at least one acid-activated sheet silicate, to a composition comprising the aforementioned sorbent, and to the preferred uses thereof.

The invention relates to a method for enriching, depleting, removing, recovering or fractionating nucleic acid molecules with the aid of a sorbent, where the sorbent includes at least one acid-activated sheet silicate. Preferred uses of such a sorbent are likewise disclosed.

The industrial and scientific importance of the separation and purification of biomolecules is continually increasing. Thus, separation processes for purifying or depleting DNA are important on the one hand for fundamental research, where genetic material must for example be isolated and purified, in order to generate genetically modified organisms. This is, however, also currently being increasingly used industrially. Thus, some of the active ingredients used in medicine are already produced by genetic manipulation.

A further field of application of such separation processes, and the adsorbents employed therefor, is represented by the depletion of DNA in wastewaters, especially associated with production processes with genetically modified organisms such as, for example, bacteria or fungi.

A large number of adsorbents are already known in the state of the art, especially those based on silanized silicate particles (silica gel) or functionalized celluloses.

U.S. Pat. No. 4,029,583 describes a silica gel chromatographic support material suitable for separating proteins, peptides and nucleic acids, which has a cavity diameter of up to 50 nm, and to which is linked by means of a silanizing reagent a stationary phase having anion or cation exchanger-forming groups which interact with the substances to be separated. The silanized silica gel is brought into contact with water, entailing the risk of the stationary phase polymerizing and the pores of the support material closing.

According to EP-B 0 104 210, nucleic acid mixtures can be fractionated into their constituents with high resolution and at a high flow rate on use of a chromatographic support material in which the diameter of the cavities amounts to one to twenty times the largest dimension of the nucleic acid to be isolated in each case or the largest dimension of the largest of all the nucleic acids present in the mixture. The chromatographic support material is produced by initially reacting it with a silanizing reagent which has a flexible chain group which in turn is converted by reaction with an anion or cation exchanger-forming reagent to the finished support material.

EP 0 496 822 (WO 91/05606, DE 393 50 98) describes a chromatographic support material whose cavities have one to twenty times the size of the largest dimension of the nucleic acids to be separated, which can be obtained by reacting a starting support material with a cavity size of from 10 to 1000 nm, a specific surface area of from 5 to 800 m²/g and a particle size of from 3 to 500 μm with a silanizing reagent which is characterized in that the silanizing reagent has at least one reactive group already reacted with a primary or secondary hydroxyalkylamine or comprises a reactive group, such as an epoxy group or halogen atoms, which can be reacted with a hydroxyalkylamine and which, in a further reaction stage, is reacted with a hydroxyalkylamine.

The article by T. G. Lawson et al., “Separation of synthetic oligonucleotides on columns of microparticulate”, Analytical Biochemistry (1983), 133(1), 85-93, describes the separation of synthetic oligonucleotides on columns based on micro-particulate silicon dioxide or silica gel which has been coated with crosslinked polyethyleneimine. The coating was in this case achieved by pumping the polyethyleneimine solution through the silica gel column. The method described in this article can be employed only for small amounts. In addition, this article refers only to polyethyleneimine-modified silica gel particles.

Further adsorbent systems are described in US 2003003272, EP 1 162 459 and EP 281 390. The article “Nukleinsäure-aufreinigung durch Kationen-Komplexierung” [Nucleic acid purification by cation complexing] by Prof. Michael Lorenz, Molzym GmbH & Co. KG, Bremen in Laborwelt No. 4/2003, page 40, describes a novel method for purifying nucleic acids with specific mini spin columns. According to the statements in the article, these are based on a matrix in which a clay mineral has been mixed with sand. Nothing is said about the nature of the clay minerals.

It is a disadvantage of the prior art sorption systems that they either are relatively costly or do not comply with requirements in the binding capacity, the kinetics of binding and/or the rate of recovery of the absorbed nucleic acid(s). Because of the increasing importance of the separation or purification of nucleic acids from various media, there is a continuing demand for improved sorbents for nucleic acids.

The present invention was therefore based on the object of providing an improved sorbent for nucleic acids which can be employed advantageously in a method for enriching or depleting, for removing or recovering, or for fractionating nucleic acids and which avoids the prior art disadvantages.

It has now astonishingly been found that to achieve this object it is possible particularly advantageously to use sorbents which include at least one acid-activated sheet silicate. Such acid-activated sheet silicates show a surprisingly high binding capacity for nucleic acids which even exceeds that of commercial prior art adsorption systems. They additionally show particularly rapid kinetics of binding. An additional advantage is that the bound nucleic acid can be removed virtually quantitatively again from the sorbent.

One aspect of the present invention thus relates to a method for sorption, enriching or depleting, removing, recovering or fractionating at least one nucleic acid molecule, preferably from a polar, in particular an aqueous or alcoholic medium with the aid of a sorbent, where the sorbent includes at least one acid-activated sheet silicate.

The sorbents disclosed herein are thus both suitable for fractionating nucleic acids and for enriching or depleting them, for recovery or removal, from appropriate solutions/media; the almost quantitative recovery rate on elution with suitable, normally high salt-content buffers shows that it is also possible to recover the bound nucleic acid again. The areas of use of such sorbents are diverse. Without this invention being restricted to the following examples, some possible applications are to be mentioned: it is conceivable for example to separate nucleic acids from a multicomponent mixture or to deplete DNA from wastewaters from biotechnological production residues with genetically modified organisms. It is possible in general for the sorbent of the invention also to be employed for all molecular biological, microbiological or biotechnological methods in connection with nucleic acids, especially the enrichment or depletion, fractionation, transient or permanent immobilization or other utilization thereof. Examples of methods and processes are to be found in relevant textbooks such as Sambrook et al., “Molecular Cloning: A Laboratory Manual”, Cold Spring Harbour Press 2001 and are familiar to the skilled worker. The present sorbent can also be employed in the context of chromatographic fractionation of nucleic acids. Nucleic acids mean in this connection primarily DNA and RNA species, inclusive of genomic DNA and cDNA and fragments thereof, mRNA, tRNA, rRNA and further nucleic acid derivatives of natural or synthetic origin of a desired length.

A further sector is represented by the fractionation of nucleic acid mixtures, for example on a matrix (support) comprising the sorbent of the invention. The adsorbents of the invention are, however, also suitable in principle for separating or purifying proteins and other biomolecules. Biomolecule means in the context of the present invention a molecule which includes as building blocks nucleotides or nucleosides (nucleobases), amino acids, monosaccharides and/or fatty acids. According to one aspect of the present invention, reference in the description to “nucleic acid (molecule)” thus also includes other biomolecules. However, the use for the sorption of nucleic acids is particularly preferred.

Starting materials which can be used for the sheet silicates employed according to the invention are all natural or synthetic sheet silicates or mixtures thereof which can be activated by an acid, i.e. in which cations in the intermediate layers can be replaced by protons. Two- and in particular three-layer silicates are preferred. Acid-activatable sheet silicates are familiar to the skilled worker and include in particular the smectic or montmorillonite-containing sheet silicates such as bentonite. It is generally possible to use both so-called naturally active and non-naturally active sheet silicates, especially di- and trioctahedral sheet silicates of the serpentine, kaolin and talc-pyrophylite group, smectites, vermiculites, illites and chlorites, and those of the sepiolite-palygorskite group such as, for example, montmorillonite, natronite, saponite and vermiculite or hectorite, beidellite, palygorskite, and mixed layer minerals. It is of course also possible to employ mixtures of two or more of the above materials. A further possibility is for the sheet silicate employed according to the invention also to comprise further constituents (also for example non-acid-activated sheet silicates) which do not impair the intended use of the acid-activated clay, especially its sorption capacity, or in fact have useful properties.

Particularly preferred sheet silicates are those of the montmorillonite/beidellite series such as, for example, montmorillonite, bentonite, natronite, saponite and hectorite. Bentonites are most preferred because in this case surprisingly particularly advantageous binding capacities and kinetics of binding for nucleic acids are achieved.

The products of the weathering of clays having a specific surface area of more than 200 m²/g, a pore volume of more than 0.5 ml/g and an ion exchange capacity of more than 40 meq/100 g in acid-activated form have also proved to be particularly useful. Raw clays whose ion exchange capacity are above 50 meq/100 g, preferably in the range from 55 to 85 meq/100 g, are particularly preferred according to this specific embodiment for the acid activation. The specific BET surface area is particularly preferably in the range from 200 to 280 m²/g, in particular between 200 and 260 m²/g. The pore volume is preferably in the range from 0.7 to 1.0 ml/100 g, in particular in the range from 0.80 to 1.0 ml/100 g. The acid activation of such raw clays can be carried out as specified in detail herein. Such clays are described for example in DE 103 56 894.8 of the same applicant, which in this regard is expressly incorporated in the present description by reference.

It has also been found in the context of the present invention that in particular the two-layer and the three-layer sheet silicates can be used advantageously even without acid activation for the sorption of nucleic acids and other biomolecules. The smectic sheet silicates (see above) such as bentonite are particularly preferred in this connection. In a further aspect of the present invention, therefore, it is possible to employ a non-activated sheet silicate instead of the acid-activated sheet silicate, or a mixture of the two as sorbent of the invention. Otherwise, the statements made in the present description apply correspondingly in relation to the method and the use of the sorbent.

In a preferred embodiment of the invention, the sorbent employed according to the invention is, however, based on at least one acid-activated sheet silicate, i.e. at least 50% by weight, preferably at least 75% by weight, more preferably at least 90% by weight, in particular at least 95% by weight or even at least 98% by weight of the sorbent of the invention consist of one (or more) acid-activated sheet silicate(s) as defined herein. In a preferred embodiment, no silica or silica gel is used. In a further preferred embodiment, the sorbent of the invention consists essentially or completely of at least one acid-activated sheet silicate. The sorbent employed according to the invention can, however, also be employed together with other sorbents appearing suitable to the skilled worker or further components, for example in the context of the method of the invention according to claim 1.

In a preferred embodiment of the invention, the acid-activated sheet silicate has an average pore diameter determined by the BJH method (DIN 66131) of between about 2 nm and 25 nm, in particular between about 4 and about 10 nm.

In a preferred embodiment of the invention, the pore volume, determined by the CCl₄ method in accordance with the methods section, of pores up to 80 nm in diameter is between about 0.15 and 0.80 ml/g, in particular between about 0.2 and 0.7 ml/g. The corresponding values for pores up to 25 nm in diameter are in the range between about 0.15 and 0.45 ml/g, in particular 0.18 to 0.40 ml/g. The corresponding values for pores up to 14 nm are in the range between about 0.10 and 0.40 ml/g, in particular about 0.12 to 0.37 ml/g. The pore volumes for pores between 14 and 25 nm in diameter may be for example between 0.02 and 0.3 ml/g. The pore volume of pores with 25 to 80 nm can be for example in the same range.

The porosimetry of the acid-activated sheet silicates can also be influenced deliberately by the conditions during the acid activation of the sheet silicates, i.e. in particular the amount and concentration of the acid employed, the temperature and the duration of the acid treatment. Thus, for example, a greater porosity of the sheet silicates can be brought about by a stronger acid activation with an increased amount of acid or at an elevated temperature over a longer period, especially in the range of smaller pores with a diameter of less than 50 nm, in particular less than 10 nm, determined by the CCl₄ method in accordance with the methods section. Thus, the micropore volume of the sheet silicate can be increased by increasing the amount of acid used for the acid activation. At the same time, the cation exchange capacity declines. It is thus possible to optimize, by routine investigation of a series of differently acid-activated sheet silicates, the sorption capacity of the acid-activated sheet silicate for the nucleic acid species of interest in each case, or its rate of absorption and desorption via the acid activation in the individual case. For example, the pores/cavities in the sorbents of the invention can be modified via the acid activation in the manner provided in EP 0 104 210 or U.S. Pat. No. 4,029,583 (see above).

The acid-activated sheet silicates employed according to the invention are generally prepared by treating sheet silicates with at least one acid. For this purpose, the sheet silicates are brought into contact with the acid(s). It is possible in this connection in principle to use any method familiar to the skilled worker for acid activation of sheet silicates, including the methods described in WO 99/02256, U.S. Pat. No. 5,008,226 and U.S. Pat. No. 5,869,415, which are to this extent expressly included in the description by reference. It is possible to use in general any organic or inorganic acids or mixtures thereof. For example, acid can be sprayed on by a so-called SMBE process (surface modified bleaching earth). The activation in this case takes place on the surface of the sheet silicates without operating in a solution or dispersion.

In a first embodiment, therefore, the activation of the sheet silicate is carried out in aqueous phase. For this purpose, the acid is brought into contact as aqueous solution with the sheet silicate. The procedure in this case can be such that initially the sheet silicate, which is preferably provided in the form of a powder, is slurried in water. Subsequently, the acid (e.g. in concentrated form) is added. However, the sheet silicate can also be slurried directly in an aqueous solution of the acid, or the aqueous solution of the acid can be put onto the sheet silicate. In an advantageous embodiment, the aqueous acid solution can for example be sprayed onto a preferably crushed or powdered sheet silicate, in which case the amount of water is preferably kept as small as possible and, for example, a concentrated acid or acid solution is employed. The amount of acid can preferably be chosen to be between 1 and 10% by weight, particularly preferably between 2 and 6% by weight of an acid, in particular of a strong acid, e.g. of a mineral acid such as sulfuric acid, based on the anhydrous sheet silicate (absolutely dry). If necessary, excess water can be evaporated off, and the activated sheet silicate can then be ground to the desired fineness. As already explained above, in this embodiment of the method of the invention a washing step is unnecessary, but possible. Putting on of the aqueous solution of the acid is merely followed, if necessary, by drying until the desired moisture content is reached. Usually, the water content of the resulting acid-activated sheet silicate is adjusted to a content of less than 20% by weight, preferably less than 15% by weight.

The acid for the activation described above with an aqueous solution of an acid or of a concentrated acid can be chosen as desired per se. It is possible to use both mineral acids and organic acids or mixtures of the aforementioned acids. Usual mineral acids can be used, such as hydrochloric acid, phosphoric acid or sulfuric acid, with preference for sulfuric acid. It is possible to use concentrated or dilute acids or acid solutions. Organic acids which can be used are solutions of, for example, citric acid or oxalic acid.

A further preferred possibility for activation is represented by boiling the sheet silicates in an acid, in particular hydrochloric or sulfuric acid. In this case, different degrees of activation can be adjusted by the suitable concentrations of acid and boiling times, and the pore volume distribution can be deliberately adjusted. Such activated sheet silicates are frequently also referred to as bleaching earths. Drying of the materials is followed by grinding thereof by conventional methods.

In the “classical” activation, which is preferred according to the invention in many cases, activation takes place at temperatures round about 100° C. to the boiling point. By contrast, the SMBE method is normally carried out at room temperature, with elevated temperatures making better acid activations possible in individual cases. The influence of the temperature in the SMBE method is, however, far less than in the “classical” activation (so-called HBPE method). The holdup time (duration of the acid activation) in the HBPE method is for example between about 8 hours, e.g. on use of hydrochloric acid, and 12 to 15 hours, e.g. on use of sulfuric acid. The HBPE method differs from the SMBE method in that the sheet structure is attacked, resulting in regions with silicic acid, in addition to areas of substantially unchanged structure. In the SMBE method, for example, 3% by weight H₂SO₄ are put on (100+3). Analysis of the worked-up material then normally reveals acid contents in the range from 0.4 to 1.0%, i.e. most of the acid is consumed (exchange of H⁺ions for other cations, etc.). A small portion is consumed where appropriate by lime which is present. In the SMBE method, the contact times with the acid are frequently about 15 minutes in the laboratory.

It has been found that, depending on the sheet silicate used, activation with small amounts of acid may suffice to obtain surprisingly good sorbents.

In a particularly preferred embodiment of the invention, the sheet silicate is activated in such a way that the cation exchange capacity (CEC) of the employed acid-activated sheet silicate is less than 50 meq/100 g, in particular less than 40 meq/100 g. The activation in this case particularly preferably takes place using an at least 1 molar, in particular at least 2 molar acid solution at elevated temperature, in particular at more than 30° C., more preferably more than 60° C. In a further preferred embodiment, an acid with a pKa of less than 4, in particular less than 3, more preferably less than 2.5., is employed for advantageous activation of the sheet silicates. Examples preferably employed are strong mineral acids, in particular hydrochloric acid, sulfuric acid or nitric acid or mixtures thereof, in particular in concentrated form. The preferred amount of acid is more than 1% by weight, in particular more than 2% by weight, particularly preferably at least 3% by weight of acid, more preferably at least 4% by weight of acid based on the amount of sheet silicate to be activated (determined after drying at 130° C.). In a particularly preferred embodiment of the invention, the exchangeable (metal) cations (intermediate layer cations) are substantially completely replaced by protons by the acid activation of the sheet silicate, i.e. to the extent of more than 90%, in particular more than 95%, particularly preferably more than 98%. This can be determined by means of the CEC and the ion contents thereof before and after the acid activation.

In one embodiment, it is unnecessary in the acid activation to wash out the excess acid and the salts formed in the activation. On the contrary, after the acid has been put on, as usual in the acid activation, no washing step is carried out, but the treated sheet silicate is dried and then ground to the desired particle size. Usually a typical bleaching earth fineness is adjusted during the grinding. In this case, the dry sieve residue on a sieve with a mesh width of 63 μm is in the range from 20 to 40% by weight. The dry sieve residue on a sieve with a mesh width of 25 μm is in the range from 50 to 65% by weight.

The sorbent employed according to the invention can be employed in the form of a powder, granules or of a shaped article of any shape. In the case of powders, use in the form of suspensions of the sorbent in the media containing the at least one nucleic acid molecule is appropriate. On the other hand, particles showing the particle size distribution usual in chromatography can also be adjusted by coarser grinding, so that the materials can also be packed into gravity columns or chromatography columns. In general, the sorbents can be used in any desired form, including supported or immobilized forms. For example, use in the fractionation of different nucleic acid components on the basis of their molecular weight is also conceivable. The form of application of the adsorbents of the invention is in this connection not restricted to the cited examples.

In general, the particle size or size of the shaped article of the acid-activated sheet silicate used as sorbent according to the invention will therefore depend on the particular application. All particle sizes or agglomerate sizes are possible in this case. For example, the acid-activated sheet silicate can be employed in powder form, in particular with a D₅₀ of from 1 to 1000 μm, in particular from 5 to 500 μm. Typical useful granules are in the range (D₅₀) between 100 μm to 5000 μm, in particular 200 to 2000 μm particle size. For many applications it is possible advantageously to have recourse to shaped articles made of or having the acid-activated sheet silicates, for example in chromatography columns, inclusive of gravity or centrifugation columns, solid-phase chromatographies, filter cartridges, membranes, etc.

In a particularly preferred embodiment of the invention it is possible, as mentioned above, for the sorbent employed according to the invention to be in immobilized form. For example, the sorbent can be incorporated in a filter cartridge, an HPLC cartridge or a comparable presentation. Incorporation in gels such as, for example agarose gels or other gelatinous or matrix-like structures is also preferably possible. Such applications are frequently sold in the framework of so-called kits for purifying nucleic acid molecules, such as, for example, the products of Quiagen, such as Quiagen genomic tip or the like. This generally entails passing the medium containing the nucleic acid molecules of interest through a column or filter cartridge or the like containing the sorbent. It is then possible to wash with suitable buffers in order to remove adherent impurities. This is finally followed by an elution step to recover the nucleic acid molecules of interest.

In a further preferred embodiment of the invention, the acid-activated sheet silicate has a BET surface area (determined as specified in DIN 66131) of at least 50 to 800 m²/g, in particular at least 100 to 600 m²/g, particularly preferably at least 130 to 500 m²/g. The large surface area evidently facilitates the interaction with the nucleic acid, with the possibility of desorption surprisingly being retained.

In a preferred embodiment of the invention, the nucleic acids are DNA or RNA molecules in double-stranded or single-stranded form with one or more nucleotide building blocks.

In relation to nucleic acids, the method of the invention is particularly advantageous in media which comprise oligo-nucleotides or nucleic acids having at least 10 bases (base pairs), preferably having at least 100 bases (base pairs), in particular at least 1000 bases (base pairs). The method of the invention can, of course, also be employed for nucleic acids of between 1 and 10 bases (base pairs) or for quite large nucleic acid molecules such as plasmids or vectors having, for example, 1 to 50 kB or longer genomic or cDNA fragments. Likewise included are restriction-digested DNA and RNA fragments, synthetic or natural oligo- and polymers of nucleic acids, cosmids, etc.

An example of interest is that the chromatographic separation of biological macromolecules such as long-chain oligonucleotides, high molecular weight nucleic acids, tRNA, 5S-rRNA, other rRNA species, single-stranded DNA, double-stranded DNA (e.g. plasmids or fragments of genomic DNA), etc. It is moreover possible with the method of the invention surprisingly to achieve an improved resolution with high flow rate. The support materials used can moreover be employed in a wide temperature range and show a high loadability. The support material also shows a great resistance to pressure and a long useful life.

There is also an increase in demand for high-purity nucleic acids such as, for example, high-purity plasmid DNA for modern biotechnological but also medical development, such as, for example, in the area of gene therapy. The protocols known in the prior art for purifying nucleic acids to high purity are frequently costly and/or time-consuming, unsuitable for use on the industrial scale or not reliable enough for therapeutic purposes, because toxic solvents or enzymes of animal origin such as, for example, RNAse are used.

The sorbent of the invention can generally be employed in any media. Polar media, in which the biomolecules or nucleic acids of interest are usually present, are preferred.

The particularly preferred aqueous or alcoholic media mean according to the invention all water- or alcohol-containing media, including aqueous-alcoholic media. Generally included are also all media in which water is completely miscible or completely mixed with other solvents. Mention should be made in particular of alcohols such as methanol, ethanol and C₃ to C₁₀ alcohols having one or more OH groups or else acids. Also conceivable are thus solvents completely miscible with water, and mixtures thereof with water and alcohol. In practice, these are in particular aqueous, aqueous-alcoholic or alcoholic media in connection with a solution, suspension, dispersion, colloidal solution or emulsion.

Typical examples are aqueous or alcoholic buffer systems like those used in science and industry, industrial or non-industrial wastewaters, process waters, fermentation residues or media, media from medical or biological research, liquid or fluid contaminated sites and the like.

The sorbent of the invention may comprise further components as long as this does not impair unacceptably the adsorption of the nucleic acids and, where intended, also the desorption thereof. Such additional components may include, without being restricted thereto, organic or inorganic binders (see below), further sorbents familiar to the skilled worker for biomolecules or other inorganic or organic substances of interest from the medium, or else support materials such as glass, plastics or ceramic materials or the like.

Thus, in an advantageous embodiment of the invention, the sorbent particles can be linked by a suitable binder to larger agglomerates, granules or shaped articles or applied to a support. The shape and size of such superordinate structures which comprise the primary sorbent particles or sheet silicate particles depends on the desired application in each case. It is thus possible to employ all shapes and sizes which are familiar to the skilled worker and suitable in the individual case. For example, in many cases agglomerates having a diameter of more than 10 μm, in particular more than 50 μm, may be preferred. Moreover, a spherical shape of the agglomerates may be advantageous for a packing for chromatography columns and the like. Examples of possible supports are calcium carbonate, plastics or ceramic materials.

It is also possible to use any binder familiar to the skilled worker as long as it does not too greatly impair the deposition or infiltration of the biomolecules into or onto the sorbent, and the stability, to be required for the particular application, of the particle agglomerates or shaped articles is ensured. Examples of binders which can be used, without restriction thereto, are: agar-agar, alginates, chitosans, pectins, gelatins, lupin protein isolates or gluten.

As already stated above, it has surprisingly been found in one aspect of the invention that the acid-activated sheet silicates themselves provide particularly favorable surfaces for the sorption of nucleic acids. It is thus preferred according to the invention for no (additional) use or treatment of the sheet silicate with cationic polymers and/or polycations (multivalent cations) to take place. It is further preferred according to the invention for no other polymers (e.g. polysaccharides), polyelectrolytes, polyanions and/or complexing agents (for modifying the sheet silicate) to be used. In a particularly preferred embodiment of the invention, in particular no cationic polymer such as, for example, an aminated polysaccharide polymer or polycation is employed. In particular, in a further preferred embodiment of the invention, the acid-activated sheet silicate used according to the invention is not modified or treated with a (cationic) polymer or a polycation.

In a further aspect, the invention relates to a method which includes the following steps:
a) contacting the preferably aqueous or alcoholic medium which comprises the at least one nucleic acid molecule with the sorbent,
b) enabling the sorption of the at least one nucleic acid molecule onto or into the sorbent,
c) separation of the sorbed or sorbent-bound nucleic acid molecule together with the sorbent from the preferably aqueous or alcoholic medium,
d) where appropriate separation or desorption of the at least one nucleic acid molecule from the sorbent.

The method of the invention for deposition or infiltration of nucleic acids onto or into the sorbent can be utilized both for enrichment (i.e. increasing the concentration of the desired nucleic acid molecule(s)) and depletion (i.e. reduction in the concentration of the desired nucleic acid molecule(s)) or fractionation of a plurality of different nucleic acid molecules.

If the method of the invention is intended to remove or dispose of nucleic acid molecules, it is possible in a further step to dispose of the sorbent comprising the nucleic acid molecules. The disposal can in this case take place for example by thermal treatment to remove the sheet silicate comprising the biomolecules, in which case the sheet silicate can be disposed of after the thermal disintegration of the nucleic acid molecules.

It is thus possible in a first aspect of the invention to remove nucleic acids deliberately from media. This plays a great part for example in wastewater treatment because in this connection strict legal regulations exist in most countries concerning the removal of nucleic acids and other biomolecules from wastewaters.

In a further preferred embodiment of the invention, it is also possible to carry out the depletion or removal of nucleic acid molecules from culture media. Thus, for example in bioreactors, it is possible for an unwanted increase in the viscosity to occur owing to the high concentration of nucleic acid molecules, in particular high molecular weight nucleic acids, present in the medium. In this case it is possible by the method of the invention to remove the interfering nucleic acid molecules from the culture medium in an efficient and biocompatible manner. The viscosity can also be adjusted to a desired extent through addition of the sorbent of the invention to the culture medium.

It is likewise desired in many cases to increase the concentration of nucleic acid molecules in a medium or to recover these nucleic acid molecules in pure form if possible. For example, the recovery or purification of desired nucleic acids from solutions is one of the standard procedures in biological and medical research. It is moreover possible according to the invention in a further step for the nucleic acid molecule to be desorbed or recovered again from the sorbent, making it possible for the sorbent also to be employed anew, where appropriate after renewed acid activation of the sheet silicate.

A further aspect of the present invention relates to a composition with a sorbent and with at least one nucleic acid molecule as defined in the present description, preferably in a polar, in particular in an aqueous or alcoholic medium.

A further aspect of the present invention relates to the use of the sorbents of the invention as inorganic vectors for introducing biomolecules into cells, or as pharmaceutical composition, in particular as reservoir for the storage and controlled release of biomolecules, preferably of nucleic acids. It has thus been found, surprisingly, that the sorbents of the invention are also suitable for efficient insertion of these biomolecules into prokaryotic or eukaryotic cells. It is evidently possible in the method of the invention for biomolecules, in particular nucleic acids, to be “packaged” in a particularly advantageous manner for insertion into cells. The principal mechanism of such an insertion for the example of DNA-LDH nanohybrids is described for example in the reference Choy et al., Angew. Chem. 2000, 112 (22), pages 4207-4211, and in EP 0 987 328 A2, to which reference is made in this regard and which is hereby included in the description by reference in relation to the method. The use as pharmaceutical composition, in particular as reservoir for the storage and controlled release of biomolecules, preferably of nucleic acids, is described as such in WO 01/49869, to which reference is made in this regard and which is hereby included in the description by reference.

### Methods section

The BET surface areas indicated herein were determined as specified in DIN 66131.

The indicated (average) pore diameters, volumes and areas were determined by using a completely automatic nitrogen adsorption-measuring apparatus (ASAP 2000, from Micrometrics) according to the manufacturer's standard program (BET, BJH, t-plot and DFT). The percentage data on the proportion of determined pore sizes relate to the total pore volume of pores between 1.7 and 300 nm in diameter (BJH Adsorption Pore Distribution Report).

Where indicated, the porosimetry was carried out by the CCl₄ method as follows:

### Reagents:

Tetrachloromethane (CCl₄)

Paraffin (liquid), from Merck, (order no. 7160.2500)

### Procedure:

1 to 2 g of the material to be tested are dried in a small weighing bottle in a drying oven at 130° C. The bottle is then cooled in a desiccator, weighed accurately and placed in a vacuum desiccator which contains the following paraffin/tetrachloromethane mixing ratios depending on the micropore volume to be measured:

| Paraffin (ml) | CCl₄ (ml) | Micropores (Å) |
|---|---|---|
| 26 | 184 | 800 |
| 47.9 | 162.1 | 390 |
| 66.5 | 143.5 | 250 |
| 82.5 | 127.5 | 180 |
| 96.4 | 113.6 | 140 |
| 108.7 | 101.3 | 115 |

The desiccator is connected to a graduated cold trap, manometer and vacuum pump and then evacuated until the contents boil. 10 ml of tetrachloromethane are evaporated and collected in the cold trap.

The contents of the desiccator are then allowed to equilibrate at room temperature for 16 to 20 hours, and subsequently air is slowly allowed into the desiccator. After removal of the desiccator lid, the weighing bottle is immediately closed and reweighed on an analytical balance.

### Evaluation:

The values are calculated in milligrams of tetrachloromethane adsorbed per gram of substance through the weight gain. Division by the density of tetrachloromethane results in the pore volume in ml/g of substance.

$$\begin{matrix}Final weight - initial weight = \frac{weight gain}{\begin{matrix}g of substance \times initial weight \times \\ density of {CCL}_{4}\end{matrix}} \\ = ml \text{/} g of substance .\end{matrix}$$

(Tetrachloromethane at 20° C., d =1.595 g/cm³)

### Measurement of the Zeta Potential

An aqueous suspension of each of the adsorbents to be investigated was prepared with dist. water. The suspension to be measured was in each case adjusted to pH 7. The zeta potential of the particles was determined according to the principle of microelectrophoresis using the Zetaphoremeter II supplied by Particle Metrix. This entailed measurement of the rate of migration of the particles in a known electric field. The particle movements taking place in a measuring cell are observed with the aid of a microscope. The direction of migration provides information about the nature of the charge (positive or negative) and the particle velocity is directly proportional to the electrical interface charge of the particles or to the zeta potential. The particle movements in the measuring cell are ascertained by means of image analysis and, after completion of the measurement, the particle paths covered are calculated and the particle velocity resulting therefrom is ascertained.

The zeta potential (stated in mV) was calculated therefrom, taking account of the suspension temperature and the electrical conductivity.

It was surprisingly found in the context of the present invention that good results can also be achieved with sheet silicates having negative zeta potential.

### Determination of the Particle Size Distribution

A Malvern Mastersizer was employed in accordance with the manufacturer's instructions for this purpose. For air determination, about 2-3 g (1 coffee spoonful) of the sample to be investigated are put in the dry powder feeder and adjusted to the correct measurement range depending on the sample (a larger weight for a coarser sample).

For determination in water, a sample (about 1 knifetipful) is put into the water bath until the measurement range is reached (a larger weight for greater coarseness) and agitated in an ultrasound bath for 5 min. The measurement then takes place.

The invention is now explained in more detail by means of the non-restrictive examples below.

### Cation Exchange Capacity (CEC)

Principle: The clay is treated with a large excess of aqueous NH₄Cl solution and thoroughly washed, and the amount of NH₄⁺remaining on the clay is determined by elemental analysis.

Me+(clay)⁻+NH₄^{+—NH}₄⁺(clay)⁻+Me+

(Me⁺=H⁺, K⁺, Na⁺, ½ Ca²⁺, ½ Mg²⁺. . . )

Apparatus: sieve, 63 μm; ground-joint Erlenmeyer flask, 300 ml; analytical balance; membrane filter funnel, 400 ml; cellulose nitrate filters, 0.15 μm (from Sartorius); drying oven; reflux condenser; hotplate; distillation unit, VAPODEST-5 (from Gerhardt, no. 6550); graduated flasks, 250 ml; flame AAS

Chemicals: 2N NH₄Cl solution; Neβler's reagent (from Merck, cat. no. 9028); boric acid solution, 2% strength; sodium hydroxide solution, 32% strength; 0.1 N hydrochloric acid; NaCl solution, 0.1% strength; KCl solution, 0.1% strength.

Procedure: 5 g of clay are sieved through a 63 μm sieve and dried at 110° C. Then exactly 2 g are weighed by differential weighing on the analytical balance into the ground-joint Erlenmeyer flask, and 100 ml of 2N NH₄Cl solution are added. The suspension is boiled under reflux for one hour. Ammonia may be evolved with bentonites having a high CaCO₃ content. It is necessary in these cases to add NH₄Cl solution until the odor of ammonia is no longer perceptible. An additional check can be carried out with a moist indicator paper. After standing for about 16 h, the NH₄⁺bentonite is filtered off on a membrane filter funnel and washed with deionized water until substantially free of ions (about 800 ml). The washings are demonstrated to be free of ions by using Neβler's reagent which is sensitive for NH₄⁺ions. The number of washes may vary depending on the type of clay between 30 minutes and 3 days. The thoroughly washed NH₄⁺clay is removed from the filter, dried at 110° C. for 2 h, ground, sieved (63 μm sieve) and again dried at 110° C. for 2 h. The NH₄⁺content of the clay is then determined by elemental analysis.

Calculation of the CEC: The CEC of the clay was determined in a conventional manner via the NH₄⁺content of the NH₄⁺clay which was ascertained by elemental analysis of the N content. The apparatus used for this was the Vario EL 3 from Elementar-Heraeus, Hanau, Del., in accordance with the manufacturer's instructions. Data are given in meq/100 g of clay (meq/100 g).

Example: nitrogen content=0.93%;

Molecular weight: N=14.0067 g/mol

$$CEC = \frac{0.93 \times 1000}{14.0067} = 66.4 meq \text{/} 100 g$$

CEC=66.4 meq/100 g of NH₄⁺bentonite

### EXAMPLES

### 1. Preparation of a Sorbent

A raw clay with a montmorillonite content of between 70 to 80% is slurried in water and purified by centrifugation. The resulting slurry is then subjected to an acid activation. This entails the concentrations being adjusted so that 56% bentonite is mixed with 44% 36% by weight hydrochloric acid and boiled at a temperature of 95 to 99° C. for 8 hours. This is followed by washing with water until the residual chloride content is less than or equal to 5% based on the solid. To analyze the residual chloride content,. 10 g of solid are boiled in 100 ml of distilled water and filtered through a fluted filter. The filtrate is titrated against silver nitrate solution to determine the residual chloride content. Finally, drying takes place until the residual moisture content is 8 to 10% by weight. The resulting final product has a weight of 430 to 520 g/l. Particularly preferred particle sizes can be adjusted by screening or additional grinding.

### 2. Characterization of the Sorbent

The characteristic data of this sorbent (adsorbent 1) and of the corresponding degree of grinding are listed in the following tables. Characterization of the surface shows that a negative zeta potential is present in solutions. The surface charge density is, however, relatively small. Values above 200 μeq/g can be achieved here with specially modified materials.

TABLE 1
Surface charge density and zeta potential

| | Surface charge | Zeta |
|---|---|---|
| | density in | potential |
| Sample | [μeq/g] | [mV] |
| Adsorbent 1 | −31 | −46.5 |

TABLE 2
Particle size distribution

| | Particle size distribution | | Particle size distribution | |
|---|---|---|---|---|
| | in air | | in water | |
| Sample | μm | [%] | μm | [%] |
| Adsorbent 1 | >25 | 4.13 | >25 | 5.41 |
| | >20 | 6.14 | >20 | 9.15 |
| | >10 | 18.56 | >10 | 30.00 |
| | <5 | 57.42 | <5 | 38.42 |
| | <2 | 27.57 | <2 | 7.61 |
| | <1 | 10.79 | <1 | 0.87 |

Adsorbent 1 was characterized by the BJH method and BET method (DIN 66131) for the average pore diameter and the BET surface area. The following values resulted:

TABLE 3
BET surface area and pore diameter

| Characteristic data | Value | |
|---|---|---|
| BET surface area | 270 | m²/g |
| Average pore diameter 4V/A, BET | 5.7 | nm |
| Average pore diameter 4V/A, BJH | 5.9 | nm |
| BJH: Cumulative pore volume for pores from | 0.42 | cm³/g |
| 1.7 to 300 nm | | |

The values resulting from the CCl₄ method (cf. above) were as follows:

TABLE 4
Pore diameter and pore volume

| Range of pore diameters (nm) | Pore volume (ml/g) |
|---|---|
| 0-14 | 0.279 |
| 14-25 | 0.032 |
| 25-80 | 0.034 |

In order to test the suitability of the novel type of adsorbent for binding DNA, adsorption experiments were carried out with herring sperm DNA (Aldrich).

To determine the concentration in the adsorption experiments, the DNA concentration was determined by photometry. A wavelength of 260 nm was set for the measurement in this case. The method was calibrated by carrying out a measurement with a series of concentrations of the DNA salt employed. The resulting calibration line was employed for photometric determination of the DNA concentration in the adsorption experiments.

For the adsorption experiments, a herring sperm DNA solution with a concentration of 1 mg/ml, 2 mg/ml, 5.63 mg/ml and 9.9 mg/l was prepared and adjusted to pH 8 with 10 mM Tris/HCl and 1 mM EDTA. Then, 0.1 g of the adsorbents was in each case mixed with 5 ml of the DNA solution and shaken at room temperature for 1 hour. This was followed by centrifugation at 2500 rpm for 15 minutes, and the supernatant was sterilized by filtration. Finally, the DNA concentration in the supernatant was measured and the DNA binding capacity was calculated therefrom. The results are compiled in the following table and in the following graph:

TABLE 5
DNA binding capacities

| DNA solution [mg/ml] | 1 | 2 | 5.63 | 9.87 |
|---|---|---|---|---|
| BC (adsorbent 1) | 11 | 30 | 116.5 | 133.5 |
| [mg DNA/g adsorbent] | | | | |

BC = Binding capacity → calculated in mg of DNA based on 1 g of the adsorbents

The bound DNA was recovered from the adsorbents by eluting with 1.5 molar sodium chloride solution in 10 mM Tris HCL pH 8.5 for 1 h (elution volume: 100 ml), centrifuging at 2000 rpm for 15 min, sterilizing the supernatant by filtration and measuring the absorption.

TABLE 4
Elution of the bound DNA

| | | Recovery rate in % |
|---|---|---|
| Concentration on | DNA recovered | of the previously bound |
| loaded adsorbent 1 | in the eluate | DNA |
| 1 mg/ml | 10.06 mg/g | 91.4% |
| 10 mg/ml | 123.5 mg/g | 92.4% |

It was found in this case that the bound DNA can be recovered again virtually quantitatively from the adsorbents. This shows the potential use of the novel adsorbents both for separating and for purifying DNA.

In order to be able to categorize the DNA binding capacity of the adsorbents of the invention compared with the prior art, analogous binding tests were carried out with a commercially available anion exchanger (Quiagen®, genomic Tip). The matrix was removed from the column and ground to a particle size comparable to the material of the invention. The comparative results are listed in the table below.

TABLE 5
Comparative results on the DNA binding capacity
with commercially available adsorbent

| | Binding capacity in mg · g⁻¹ |
|---|---|
| Adsorbent | after 16 h with 2.5 mg/ml DNA |
| Weakly basic anion exchanger | 12.6 |
| (Quiagen ®) | |

As comparison of table 3 and table 4 shows, the binding capacity of the adsorbent type of the invention is considerably higher than that of the comparative anion exchanger. The binding capacities of adsorbents commercially available according to the prior art are thus reached or exceeded. An additional factor is that the adsorbents of the invention display substantially faster DNA binding because the corresponding amounts of DNA are bound after only 1 hour compared with the adsorption time of 16 hours with the comparative material.

The data suggests that the binding sites of the adsorbents of the invention are substantially better accessible, especially for large biomolecules, than for the comparative adsorbent.

4. Transfection of NIH-3T3 Cells with Acid-Activated Sheet Silicate as Inorganic Vector

### a) Cell Cultivation:

The NIH-3T3 cell line was used for the transfection experiments. This takes the form of adherently growing mouse embryo fibroblasts. The doubling time is about 20 h. The cells are cultivated in the standard medium DMEM (with 4.5 g·1⁻¹ glucose) with 10% NCS. Cultivation takes place in an incubator at 37° C. under a humidified 5% CO₂ atmosphere.

To set up the stock culture, the thawed cell suspension is put in a monolayer flask (25 cm²) with 10 ml of medium. A direct determination of the cell count is impossible with adherently growing cells, and the growth rate is checked via the degree of coverage of the culture vessel. At about 90% confluence —usually after 3-4 days—the culture is transferred in order to avoid overgrowth of the cells (formation of foci). For this purpose, the medium is decanted off, and trypsin solution is added and incubated in an incubator for 10 min. The detached cell suspension is mixed with about 15 ml of serum-containing medium in order to block the trypsin. The centrifuged cells are resuspended in fresh medium and seeded anew in a dilution of 1: 10.

### b) Transfection:

The adsorbent 1 samples indicated at the outset were “loaded” with the plasmid pQBI25-fC1 (from Quiagen, Heidelberg) before the transfection as inorganic vector for transfection of NIH-3T3 cells. For this purpose, 10 mg of the respective adsorbent 1 sample were weighed out and washed with ethanol to sterilize. Subsequently, 2 ml of sterile distilled water were added, briefly vortexed and centrifuged at 5000 rpm for 3 min. 1.5 ml of sterile plasmid DNA in a concentration of 1.45 ml·ml⁻¹ were added to the residue and shaken at 250 rpm at room temperature for 16 h.

The DNA-adsorbent 1 hybrid was suspended in 10 ml of distilled sterile water. Transfection with adsorbent 1 as vector was carried out in each case with two different concentrations of DNA-adsorbent 1 hybrid. The NIH-3T3 cells were seeded at a density of 1-2·10⁵ cells·cm⁻² in 6-well plates 24 h before the transfection and incubated at 37° C. under a humidified 5% CO₂ atmosphere in an incubator. The stated amounts are based in each case on one well of the 6-well plate. Analysis took place 48 h after starting the transfection using a fluorescence microscope. Transfected cells are identified by the GFP production; on excitation at 474 nm they show green fluorescence on inspection under a fluorescence microscope.

### Variant 1:

Immediately before the experiment, the medium was removed and 1.5 ml of new medium were added. 25 or 100 μl of the DNA-adsorbent 1 suspension were added and incubated in an incubator for 3 h. The medium was then changed and incubated for a further 45 h.

### Variant 2:

Immediately before the experiment, the medium was removed and 1.5 ml of new medium were added. 25 or 100 μl of the suspension were added and incubated for 24 h. The medium was then changed and incubated for a further 24 h.

On use of the hybrid of the invention with adsorbent 1 it was possible to detect numerous successfully transfected cells on the basis of the fluorescence, so that the DNA-adsorbent 1 hybrids of the invention are suitable as inorganic vectors.

1. A method for sorption, enriching or depleting, removing or recovering or fractionating at least one nucleic acid molecule from a medium with the aid of a sorbent, where the sorbent comprises at least one acid-activated layer silicate comprising contacting said at least one nucleic acid molecule with the sorbent.

2. The method as claimed in claim 1, characterized in that the layer silicate is selected from the group consisting of natural and synthetic layer silicates and mixtures thereof.

3. The method as claimed in claim 1, characterized in that the layer silicate is not treated with a cationic polymer or polycation.

4. The method as claimed in claim 1, characterized in that the cation exchange capacity of the acid-activated layer silicate is less than 50 meq/100 g.

5. The method as claimed in claim 1, characterized in that the acid-activated layer silicate has a BET surface area of at least 50 m²/g.

6. The method as claimed in claim 1, characterized in that the acid-activated layer silicate, preferably in powder form, has a particle size (D₅₀) of from 1 to 1000 μm.

7. The method as claimed in claim 1, characterized in that the acid-activated layer silicate, preferably in granule form, has a particle size (D₅₀) of from 100 to 5000 μm.

8. The method as claimed in claim 1, characterized in that the acid-activated layer silicate has a porosimetry as follows: pores up to 80 nm in diameter between about 0.15 and 0.80 ml/g, pores up to 25 nm in diameter between about 0.15 and 0.45 ml/g, and pores up to 14 nm between about 0.10 and 0.40 ml/g, in each case determined by the CCl₄ method.

9. The method as claimed in claim 1, characterized in that the average pore diameter by the BJH method of the acid-activated layer silicate is between 2 and 25 nm.

10. The method as claimed in claim 1, characterized in that the at least one nucleic acid molecule is selected from the group consisting of mono-, oligo- and polynucleotides and mixtures thereof.

11. The method as claimed in claim 1, characterized in that the at least one nucleic acid molecule is selected from ribonucleic acids (RNA) and deoxyribonucleic acids (DNA) and mixtures thereof.

12. The method as claimed in claim 1, characterized in that the at least one nucleic acid molecule comprises at least 10 nucleotide building blocks.

13. The method as claimed in claim 1, characterized in that the aqueous or alcoholic medium with the at least one nucleic acid molecule comprises an aqueous or alcoholic medium in the form of a colloidal solution, suspension, dispersion, solution or emulsion.

14. The method as claimed in claim 1 further comprising
a. enabling the sorption of the at least one nucleic acid molecule onto or into the sorbent,
b. separating the sorbed or sorbent-bound nucleic acid molecule together with the sorbent from the preferably aqueous or alcoholic medium, and
c. separating or desorbing the at least one nucleic acid molecule from the sorbent.

15. The method as claimed in claim 1, characterized in that the sorbent consists essentially of at least one acid-activated sheet layer silicate.

16. The method as claimed in claim 1, characterized in that the acid activation of the layer silicate comprises contacting the layer silicate with an inorganic or organic acid.

17. The method as claimed in claim 1, wherein the acid activation of the layer silicate utilizes an amount of acid (anhydrous) from 1 to 35% by weight based on the sheet silicate, preferably at elevated temperature.

18. The method as claimed in claim 1, characterized in that the medium is selected from the group consisting of a DNA or RNA solution, a fermentation medium, a fermentation residue from cell culture, process water, wastewater and mixtures thereof.

19. The method as claimed in claim 1, further comprising disposing of the sorbent together with the nucleic acid molecule.

20. The method as claimed in claim 1, further comprising desorbing the at least one nucleic acid molecule from the sorbent, making it possible to employ the sorbent anew.

21. The method as claimed in claim 20, characterized in that the desorption of the at least one nucleic acid molecule takes place in a high-salt buffer solution.

22. A composition comprising the sorbent of claim 1 and at least one nucleic acid molecule blended in a preferably aqueous or alcoholic medium.

23. (canceled)

24. (canceled)

25. (canceled)

26. (canceled)

27. (canceled)

28. (canceled)

29. (canceled)

30. The method as claimed in claim 1, characterized in that the sorbent is in the form of particles and is linked with a binder to particle aggregates or shaped articles or is applied to a support.

31. The method as claimed in claim 30, characterized in the binder is selected from the group consisting of alginate, agar-agar, chitosans, pectins, gelatins, lupin protein isolates, gluten and mixtures thereof.

32. The composition as claimed in claim 22, characterized in that the sorbent is in the form of particles and is linked with a binder to particle aggregates or shaped articles or is applied to a support.

33. The composition as claimed in claim 32, characterized in that the binder is selected from the group consisting of alginate, agar-agar, chitosans, pectins, gelatins, lupin protein isolates, gluten and mixtures thereof.
